(19) United States
(12) Patent Application Publication
(10) Pub. No.: US 2018/0299433 A1
(43) Pub. Date: Oct. 18, 2018

(54) SAMPLE COLLECTION DEVICE AND METHOD FOR URINE AND OTHER FLUIDS

(71) Applicant: Wellmetris, LLC, Keego Harbor, MI (US)

(72) Inventor: Andrew A. Dahl, Keego Harbor, MI (US)

(21) Appl. No.: 15/569,376

(22) PCT Filed: Apr. 28, 2016
(86) PCT No.: PCT/US2016/029725

§ 371 Date: Oct. 25, 2017

Related U.S. Application Data
(60) Provisional application No. 62/154,892, filed on Apr. 30, 2015.

(51) Int. Cl. G01N 33/52; G01N 1/10

(52) U.S. Cl. CPC G01N 33/521; G01N 2001/002; G01N 1/10

(57) ABSTRACT

A tubular sample collection device, including a tubular housing having a top wall defining at least one perforation for admitting a sample of a fluid and a bottom wall for receiving a test strip, and a wick affixed to an interior side of the top wall for absorbing fluid admitted through the at least one perforation. A method of collecting a fluid and transferring the fluid to a test strip, by exposing the tubular sample collection device to a fluid to be analyzed, admitting a sample of the fluid through at least one perforation defined in the top wall of a tubular housing of the tubular sample collection device, absorbing at least a portion of the sample of fluid into a wick affixed within the tubular housing, contacting the wick to a test strip within the tubular sample collecting device, and transferring fluid from the wick to the test strip.

### BACKGROUND OF THE INVENTION

### 1. Technical Field

The present invention relates to devices for collecting urine and other fluids, and in particular for devices including a self-contained sampling wick and test strip, the sampling wick being depressible to contact the test strip or sensor surface.

### 2. Background Art

Urine and other body fluids are frequently collected for analysis, for purposes of medical diagnosis and wellness monitoring. In many types of analysis, a sample of collected fluid is applied to a test strip. The fluid reacts with analytic reagents or sensors that are either present in the test strip, or introduced at a later point in time. It is important to deliver a controlled amount of fluid to the test strip, to provide sufficient sample without over-saturating the strip and swamping the reagents. In many cases, it is also important to control the zero time point of reaction between the fluid and the test strip, that is, the moment at which the fluid sample comes into contact with the test strip or sensor.

In the case of urinalysis, one mode of collection is to apply urine directly to a test strip. A sample of urine is typically collected from a urine stream, in a collection vessel, such as a jar, bottle, or plastic bag. The urine is then contacted with the test strip, either by dipping the test strip into the collection vessel, or by pipetting a portion of the fluid onto the test strip. This mode of fluid collection and application to a test strip is inefficient and unsanitary. A large vessel is required to hold the product of a full or partial voiding, far more than is generally required for analysis. Spillage of urine and over-saturation of the test strip are likely to occur. A great deal of structural material is wasted in a large collection vessel, which is usually discarded. Furthermore, there is no capacity for delaying and controlling the zero time point of reaction between the urine and reagents in the test strip. Reaction commences at the moment of urine collection.

Attempts have been made to render the urine collection process more efficient and sanitary. For example, U.S. Pat. No. 6,212,698, to Stingley, et al., discloses a urine collection kit that includes a plastic bag affixable to a toilet bowl rim, with a collection cup situated at a drain at the base of the bag. The kit does not overcome the problem of having to collect a large volume of urine in a large, wasteful, disposable collection vessel. U.S. Patent Application Publication No. 2005/0004538, to Forte, discloses a urine collection device that includes a funnel-shaped vessel to catch a flow of urine, and diagnostic test strips affixed to one or more of the urine-exposed surfaces of the vessel. The device gives no control over the quantity of urine delivered to the test strips, and no control over the zero time point of reaction. It also requires a large funnel-like vessel that is discarded after use.

Fluid collection devices are also known that collect a urine sample on an absorbent wick or membrane, for transfer onto a test strip. U.S. Pat. No. 6,140,136 to Lee discloses an analytical test device including an absorbent sample pad, which can be extended out of a housing, exposed to a urine stream, and retracted back into the housing, where it comes into contact with a membrane containing test reagents. The device disclosed by Lee does allow the zero time point of reaction to be delayed beyond the time of urine collection, but only by keeping the soaked sample pad in the extended position, external to the housing. In an external position, the wick is susceptible to contamination and evaporation, and is a potential biohazard. The device of Lee also provides no control over the pressure and duration of contact between the absorbent sample pad and membrane, allowing a user no control over the amount of urine delivered to the membrane.

Similarly, U.S. Pat. No. 7,294,502 to Eckermann, et al. discloses a device for collecting samples of saliva and other body fluids. The device includes two hinged parts, one part including an absorbent pad, and the other including a test strip. In an open condition, the part including the absorbent pad is exposed, and the pad can be used to sample a body fluid. In the closed position, the parts are snapped together, and the absorbent pad is held against the test strip with a constant pressure. Again, delay of the zero time point of reaction requires that the soaked absorbent pad remain exposed to the environment, and there is no control over the pressure and duration of contact between the absorbent sample pad and the test strip.

There is a need for a device for the sanitary and efficient collection of a sample of urine or other fluid, which provides control of the duration and pressure of contact of an absorbent pad or wick to a test strip, for the application of a controlled volume of the sample, as well as control of the zero time point of reaction between the urine and the test strip, without requiring the exposure of sample-filled pad or wick to the environment or inadvertent contact by testing personnel.

### SUMMARY OF THE INVENTION

The present invention provides a tubular sample collection device, including a tubular housing having a top wall defining at least one perforation for admitting a sample of a fluid and a bottom wall for receiving a reversibly insertable test strip, and a wick affixed to an interior side of the top wall for absorbing fluid admitted through the at least one perforation.

The present invention also provides a method of collecting a fluid and transferring the fluid to a test strip, by exposing a tubular sample collection device to a fluid to be analyzed, admitting a sample of the fluid through at least one perforation defined in the top wall of a tubular housing of the tubular sample collection device, absorbing at least a portion of the sample of fluid into a wick affixed within the tubular housing, contacting the wick to a test strip within the tubular sample collecting device, and transferring fluid from the wick to the test strip.

The present invention further provides an extendable sample collection device, including an elongated wick having an extended position from an elongated housing to be exposed to and absorb a fluid sample and a retractable position in said elongated housing, the elongated housing including a top wall being depressible to bring the elongated wick in the retracted position into contact with a test strip situated within the elongated housing.

The present invention still further provides a method of collecting a fluid to be analyzed, and transferring the fluid to a test strip, by extending an elongated wick from a retracted position within in an elongated housing of an extendable sample collection device to an extended position exterior to elongated housing, exposing the elongated wick to a fluid to be analyzed, absorbing a sample of the fluid to be analyzed into the elongated wick, retracting the elongated wick into the elongated housing, contacting the elongated wick to a test strip within the elongated housing, and transferring fluid from the elongated wick to the test strip.

The present invention provides for a double biological barrier, including an inner sleeve for receiving a sample collection device, the inner sleeve fitting snugly with the sample collection device, and an outer sleeve fitting over the inner sleeve.

The present invention provides for a method of using a double biological barrier, by inserting a sample collection device in an inner sleeve of a double biological barrier, inserting the inner sleeve in an outer sleeve of the double biological barrier, and preventing transfer of fluid pathogens to users.

### DESCRIPTION OF THE DRAWINGS

Other advantages of the present invention are readily appreciated as the same becomes better understood by reference to the following detailed description when considered in connection with the accompanying drawings wherein:

FIG. 1 shows an oblique perspective view of a tubular sample collection device according to the present invention;

FIG. 2 shows a test strip in the process of insertion into a housing of the tubular sample collection device;

FIG. 3 shows a cross section of the tubular sample collection device;

FIG. 4A shows a cross section of the tubular sample collection device, including a test strip supported by a carrier;

FIG. 4B shows a top wall of the tubular sample collection device, in the process of depression to bring a wick into contact with the test strip;

FIG. 5 shows an oblique perspective view of an exemplary tubular sample collection device having perforations on the top wall, side walls, and bottom wall; also shown is a test strip, supported by a carrier, in the process insertion into the tubular housing;

FIG. 6A shows a side elevation of the tubular sample collection device;

FIG. 6B shows a bottom elevation of the tubular sample collection device;

FIG. 7A shows an end of the housing of the tubular sample collection device, the end being partially occluded by a bar;

FIG. 7B shows three alternative styles for the ends of the housing of the tubular sample device;

FIG. 8 shows a side perspective view of the tubular sample collection device, with a test strip fully inserted into the housing;

FIG. 9 shows an end perspective view of the tubular sample collection device, including a test strip supported by a carrier;

FIG. 10 shows a cutaway perspective view of a side wall of the housing of the tubular sample collection device;

FIG. 11 shows a cutaway perspective view of a side wall of the housing of the tubular sample collection device, including a fully inserted test strip;

FIG. 12A shows an exploded view of the tubular sample collection device, showing two-piece construction;

FIG. 12B shows a cross sectional exploded view of the tubular sample collection device, showing two-piece construction;

FIG. 13A shows an oblique perspective view of an extendable sample collection device according to the present invention, with arrow A indicating the directions of extension and retraction of an elongated wick relative to an elongated housing; arrow B indicating the direction of insertion of a carrier and a test strip into the elongated housing; and arrow C indicating the direction of pressure required to depress a top wall of the elongated housing;

FIG. 13B shows a bottom elevation of the extendable sample collection device, with the elongated wick in fully extended position;

FIG. 13C shows an exploded view of the extendable sample collection device;

FIG. 13D shows a front elevation of a front wall of the housing of the extendable sample collection device;

FIG. 14A shows a an oblique perspective view of the tubular sample collection device surrounded by a double biological barrier according to the present invention, with spaces between layers of the double biological barrier exaggerated for clarity;

FIG. 14B shows a bottom elevation of the extendable sample collection device surrounded by the double biological barrier, with spaces between layers of the double biological barrier exaggerated for clarity;

FIG. 15 is a top view of a test strip;

FIG. 16 is a side perspective view of the double biological barrier and sample collection device;

FIG. 17A shows a see through side view of the extendable sample collection device;

FIG. 17B shows a cross-sectional view of FIG. 17A along line A-A;

FIG. 17C shows a see through side view of the extendable sample collection device;

FIG. 17D shows a see through side view of the extendable sample collection device; and

FIG. 17E shows a see through perspective view of the extendable sample collection device.

### DETAILED DESCRIPTION OF THE INVENTION

The present invention generally provides for sample collection devices, preferably to be used for collecting and analyzing a fluid sample. The sample collection device not only collects a sample (preferably urine), but also transfers the sample to an assay strip (preferably a urinalysis assay strip) inside the device itself. This is an improvement over prior art sample collection devices because the device of the present invention collects the sample and transfers it in a neat, precise, and pre-measured manner to the assays on the test strip.

A sample collection device according to the present invention includes a housing to receive an insertable test strip, and an absorbent wick to absorb a fluid to be sampled. The absorbent wick is a two-position wick. That is, after the wick has absorbed a fluid, it resides within the housing, but is not in contact with the test strip. Instead, it is situated in an outward position, between the test strip and a depressible wall of the housing. The fluid-bearing wick remains out of contact with the test strip until the depressible wall is depressed by external pressure. The fluid-bearing wick then moves to an inward position, to make contact with the test strip, and it remains in contact until the external pressure is removed from the deformable wall.

The depression of the deformable wall can be performed with pressure applied by a human finger or robotic appendage, providing a high level of control of the magnitude and duration of pressure, and therefore of the amount of sample delivered to the test strip. The zero time point of the reaction between the fluid and the test strip can be delayed by delaying the depression of the wall. During this delay period, the fluid-bearing wick remains enclosed within the housing, safe from contamination and evaporation, and posing no external biohazard to users. In the case of urine collection, the collection device or wick can be held directly under a urine stream, eliminating the need for bulky and wasteful collection vessels.

Two exemplary embodiments of the sample collection device are disclosed: a tubular sample collection device generally shown at 10, which includes a fixed wick 24; and an extendable sample collection device generally shown at 60, which includes an elongated wick 42 that can be moved into and out of an elongated housing 30, much like the blade of a utility knife.

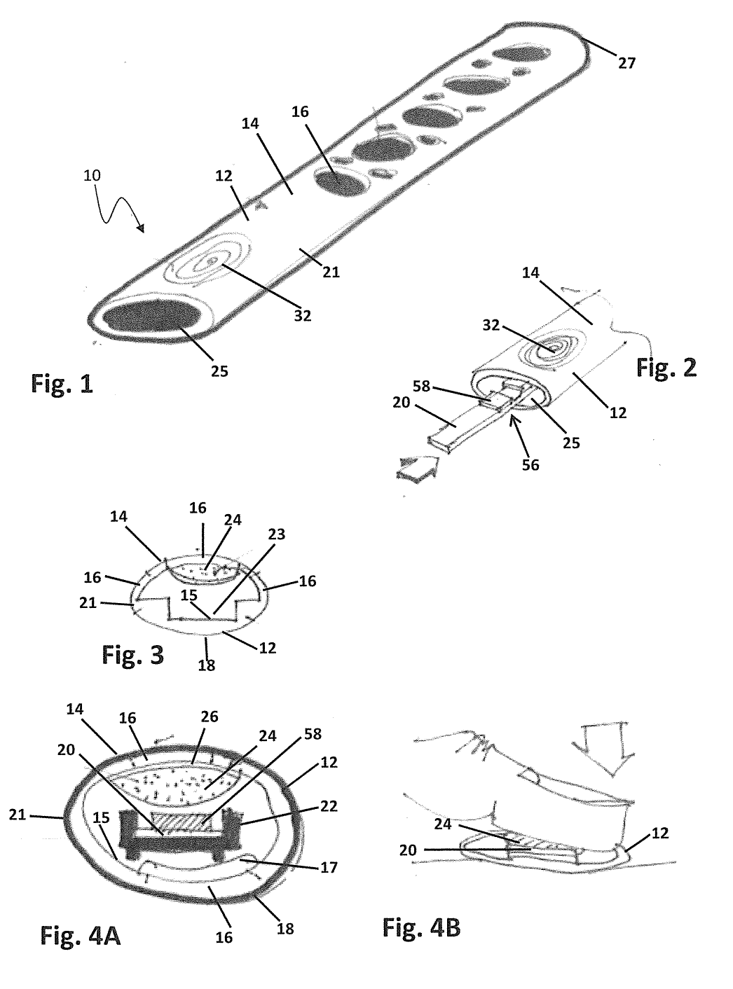

A tubular sample collection device 10, shown in FIGS. 1-12, includes a tubular housing 12, preferably elliptical in cross section, having a top wall 14 including and defining at least one perforation 16, and preferably a plurality of perforations 16, for admitting a sample of urine or other fluid sample, a bottom wall 18, for receiving a reversibly insertable test strip 20, preferably mounted on a carrier 22; and two sidewalls 21 interconnecting the top wall 14 and bottom wall 18. As shown in FIG. 10, a wick 24 is affixed to an interior side 26 of the top wall 14, to occlude the perforations 16, and absorb fluid admitted through the perforations 16. Additional perforations 16 are preferably also defined in the side walls 21 and bottom walls 18, to provide ventilation and drainage of excess fluid, as shown in FIGS. 6A and 6B.

The wick 24 can be affixed to the top wall 14 by an adhesive layer 26, which can include any suitable adhesive material or adhesive tape, including a colorimetric adhesive. The term “colorimetric adhesive” refers to any glue or tape that contains dry chemistry activatable to change color upon saturation with urine. The color change is useful to inform a user that collection can cease, and the urine stream can be directed away from the collection device 10 and into a urinal or toilet. Any suitable dry chemistry system activatable to change color by saturation with urine or other fluid can be utilized in the colorimetric adhesive.

In an exemplary embodiment, the tubular housing 12 is approximately 6 inches in length. Although the perforations 16 are depicted as round in FIGS. 1, 5, and 6, the perforations 16 alternatively can be of any desired shape and of any required size.

A test strip 20 is situated on the interior side 15 of the bottom wall 18 of the tubular housing 12. The top wall 14 of the tubular housing 12 is sufficiently depressible to maintain the wick 24 in one of two positions, an outward position wherein the wick 24 is not in contact with the test strip 20, as shown in FIGS. 4A and 11, and a depressed, inward position, wherein the wick 24 is in contact with the test strip 20, as shown in FIG. 4B. The transition from the outward to the inward position is mediated by pressure exerted upon the top wall 14 of the housing 12. Pressure is preferably applied by the finger of a user against a pressure tab 28 situated on the outer surface of the top wall, but it can also be applied mechanically, for example by robotic means, after insertion of the tubular sample collection device 10 into an automated processor (not shown). The pressure tab 28 serves to focus inward pressure at a desired area of the wick 24, usually the area opposite a detection zone 56 of the test strip 20. The detection zone 56 is the area of the test strip 20 that contains one or more analytic surfaces or sensors 58, including but not limited to, papers, filter pads, fiber pads, membranes, wells, or microchips loaded with chromatic or fluorescent reagents or adsorbed antigens, antibodies, or nucleic acid probes. The analytic surfaces 58 can be 5 mm by 5 mm pads impregnated with various chemical reagents. In an exemplary embodiment, the wick 24 and the test strip 20 extend for approximately ⅔ the length of the tubular fluid collection device 10. A test strip 20 can include multiple diverse analytic surfaces 58, differing with respect to physical materials, chemical reagents, or both.

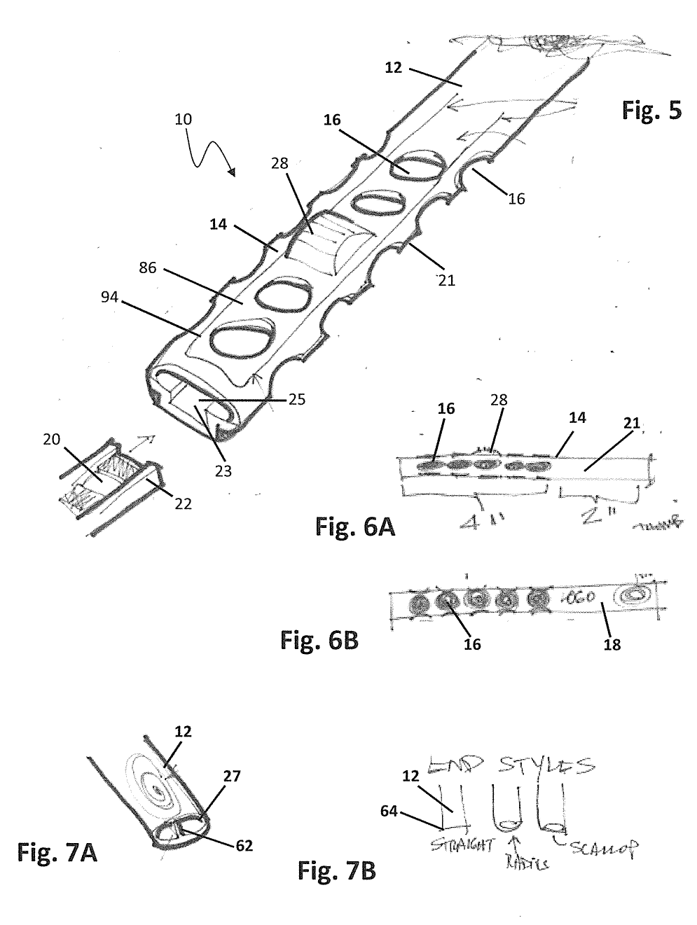

The test strip 20 is admitted into the tubular housing 12 through an open first end 25 of the tubular housing 12, as shown in FIGS. 2 and 5. An opposite second end 27 of the tubular housing 12 is generally open, but, can optionally include a bar 62, to partially occlude the second end 27, to prevent insertion of the test strip 20 at that end (FIG. 7A). The edges 64 of the first end 25 and second end 27 have a straight profile, as shown in FIG. 7B, left-hand drawing, but the edges can alternatively be scalloped, as shown in FIG. 7B, right-hand drawing, or radius, as shown in FIG. 7C, center drawing.

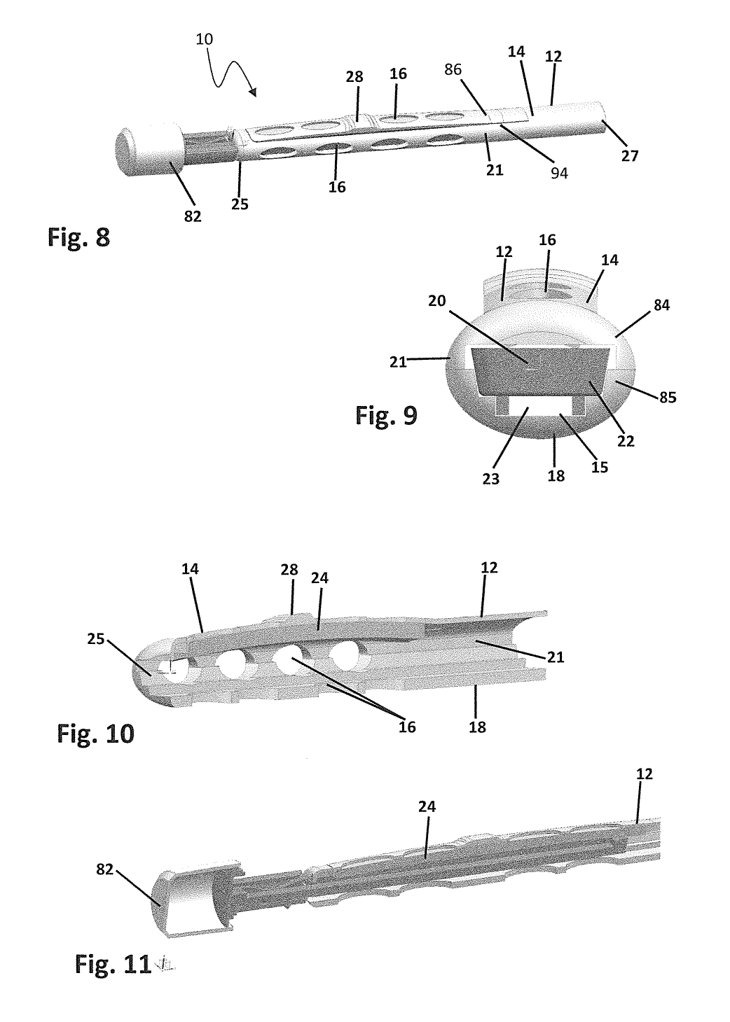

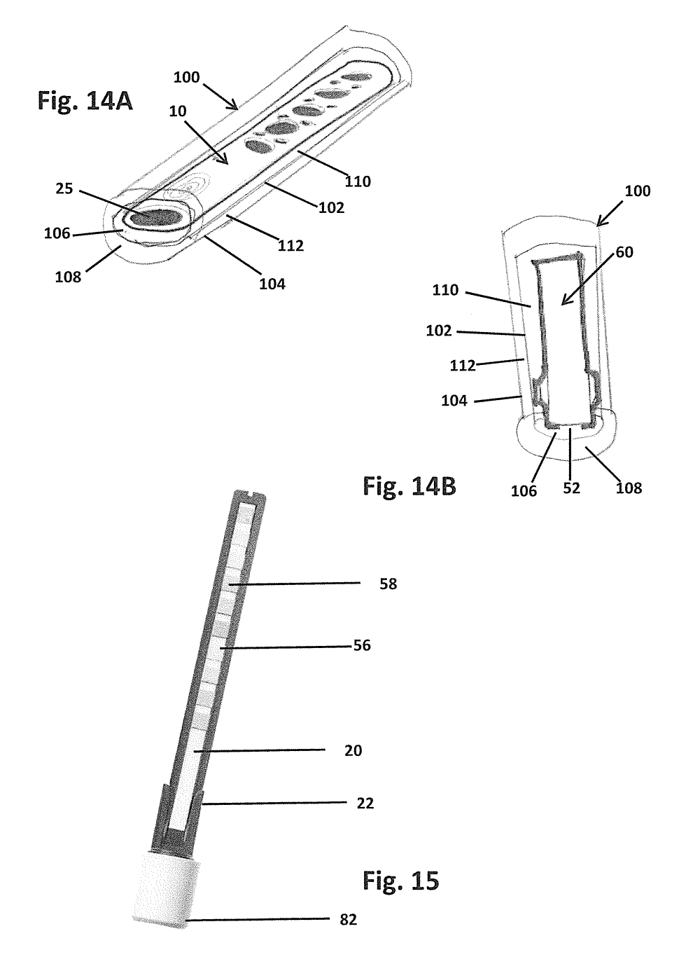

Preferably, the test strip 20 is supported by a carrier 22 (shown in FIG. 15), which facilitates the removal of the test strip 20 from the tubular sample collection device 10, and provides a solid substrate for the test strip 20 within the tubular sample collection device 10. The carrier 22 can rest directly on the bottom wall 18 of the tubular sample collection device 10, as shown in FIG. 4A. More preferably, the carrier 22 is nestable in a carrier track 23 defined in the tubular housing 12, such as the side walls 21 and/or bottom wall 18 of the tubular sample collection device 10, as shown in FIGS. 3 and 9. The carrier 22 can include a handle 82 to facilitate manipulation of the carrier 22, as shown in FIGS. 8 and 11. The handle 82 can be adapted for additional functions. For example, when used in conjunction with a test strip analyzer (not shown), the handle 82 can serve as a light shield to prevent extraneous light from entering the analyzer after insertion of the carrier 22 and test strip 20. The carrier 22 is preferably disposable.

In the preferred embodiment, the test strip 20 is removed after transfer of fluid has been accomplished, for further processing or analysis. In this embodiment, the carrier 22 is withdrawn from the tubular sample collection device 10 and inserted into a separate analyzer device (not shown) that measures the color (i.e. a chromatic change), enzymatic, or electrochemical values of each of the analytic surfaces 58 on the test strip 20, and transmits the resulting colorimetric, enzymatic, or electrochemical data to reporting software residing on a personal computer, laptop, tablet or smart phone or other suitable processing device (not shown). Embodiments wherein the color of the test strip 20 is not inserted into an analyzer, but rather is read by visual inspection, are less preferable, but are also within the scope of the present invention.

It is desirable that no residual urine or other fluid be transported on the underside of the carrier 22 when it is removed from the sample collection device 10. To remove residual fluid, the tubular sample collection device 10 optionally includes a cleaner/deodorant pad 17 affixed to the bottom wall 18, inferior to (i.e. below) the carrier 22. The cleaner/deodorant pad 17 includes a suitable quantity of any suitable cleaning agent, sealed within a thin membrane (not shown). A deodorant compound can also be included. The membrane (not shown) is breakable by the pressure applied to depress the wick 24 into contact with the test strip 20, as previously described. Breaking of the membrane (not shown) releases a quantity of cleaner to wipe the carrier 22 as it is removed from the tubular housing 12.

In embodiments adapted for direct collection of urine samples from a urine stream, an indicator 32 can be included on the tubular housing 12 to indicate which end to hold during urination.

The tubular housing 12 is preferably constructed of a lightweight, flexible hydrophobic material, most preferably a thermoplastic such as polystyrene or polypropylene. The material must be flexible enough to permit sufficient depression to bring the wick 24 into the inward position, that is, into contact with the test strip 20. It is also greatly preferred that the material be sufficiently resilient to return the wick to the outward position, out of contact with the test strip, upon release of external pressure.

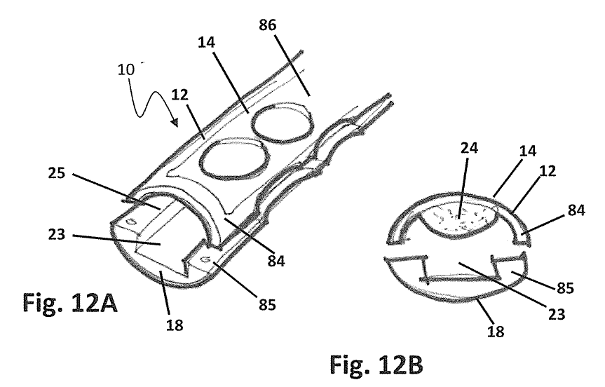

The tubular housing 12 is preferably assembled from at least two component injected molded parts. In one example, shown in FIGS. 12A and 12B, the tubular housing 12 is assembled as an upper part 84, which includes the top wall 14 and a portion of the side walls 21; and a lower part 85, which includes the remainder of the side walls 21 and the bottom wall 18. In another example, shown in FIG. 5, the top wall is partially cut to define a tongue 86, which includes the perforations 16, the wick 24, and the pressure tab 28. A three-sided cut 94 that defines the tongue 86 facilitates the inward movement of the wick 24 into contact with the test strip 20. The upper part 84 and lower part 85 of the tubular sample collection device 10 can be joined by welds, adhesives, hardware such as rivets or nuts and bolts, by a snap fit, or by any other suitable fastening means known in the art (not shown).

The present invention also provides a method of collecting a fluid to be analyzed, and transferring the sample to a detection zone 56 of a test strip 20, including the steps of exposing a tubular sample collection device 10 to a fluid to be analyzed; admitting a sample of the fluid through at least one perforation defined in the top wall 14 of a tubular housing 12 of a tubular sampling device 10; absorbing at least a portion of the sample of fluid into a wick 24 situated and affixed within the tubular housing 12, the wick 24 being in an outward position, distant from a detection zone 56 of a test strip 20 situated within the tubular housing 12; depressing the top wall 14 of the tubular housing 12; bringing at least a portion of the wick 24 into an inward position, contacting the wick 24 with the detection zone 56 of the test strip 20; and transferring at least a portion of the absorbed sample of the fluid from the wick 24 to the detection zone 56 of the test strip. The transferring step is preferably followed by the additional steps of producing a chromatic change at the at least one analytic surface 58 of the test strip 20, removing the test strip from the tubular sample collection device 10, and measuring a colorimetric, enzymatic, or electrochemical result. The step of measuring a colorimetric, enzymatic, or electrochemical result preferably includes the steps of inserting the test strip 20, preferably in the carrier 22, into an analyzing device (not shown); reading a colorimetric, enzymatic, or electrochemical value; producing colorimetric, enzymatic, or electrochemical value data; and transmitting the colorimetric, enzymatic, or electrochemical value data to reporting software residing on a personal computer, tablet, smartphone, or other suitable processing device (not shown).

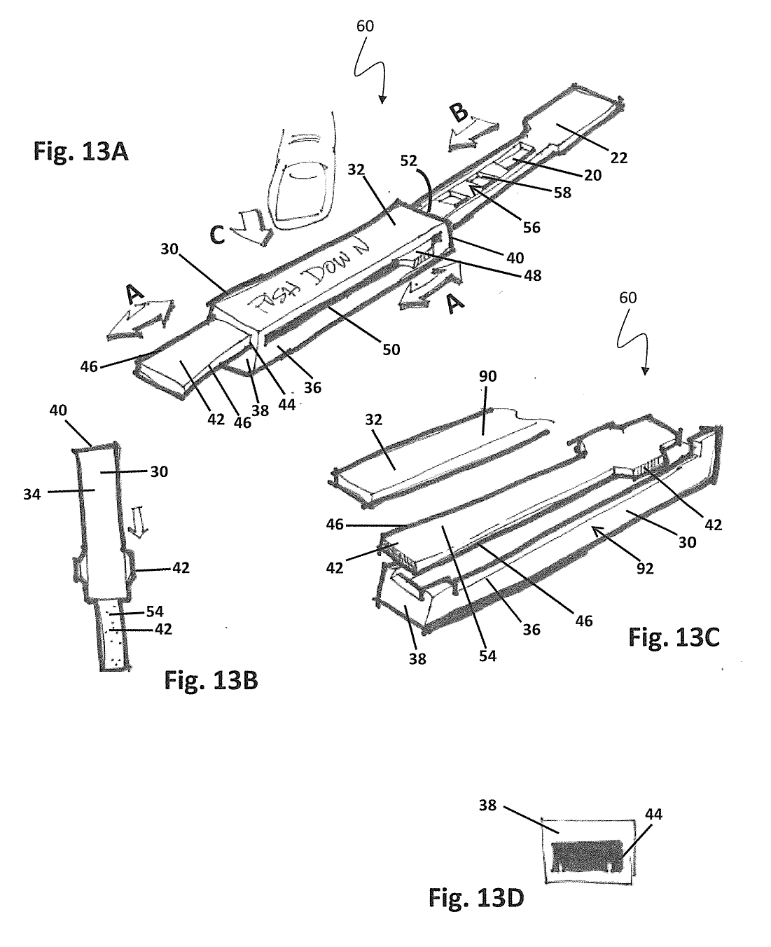

A second embodiment of the present invention is an extendable sample collection device with a movable wick, generally shown at 60 in FIG. 13. The extendable sample collection device 60 includes an elongated wick 42 that can be extended (i.e. in an extended position) from an elongated housing 30 to absorb a fluid sample, and retracted back into the housing 30 (i.e. in a retracted position). The top wall 32 of the elongated housing 30 is depressible to bring the retracted elongated wick 42 into contact with a test strip 20.

In the preferred embodiment of the extendable sample collection device 60, the elongated housing 30 is a hollow rectangular solid shape, as shown in FIGS. 13A-C and 17A-17E. The long walls of the rectangular solid include a top wall 32, a bottom wall 34, and two opposite side walls 36. The short walls of the rectangular solid include a front wall 38, and an opposite rear wall 40. A generally rectangular elongated wick 42 is situated within the elongated housing 30. The elongated wick 42 partially protrudes through, and is slidingly engaged with, a front aperture 44 defined in the front wall 38. The elongated wick 42 has two opposite long sides 46. Each long side 46 includes an outward-protruding tab 48. Each tab 48 protrudes through, and is slidingly engaged with, a slot 50 defined along at least part of the length of each of the side walls 36 of the elongated housing 30. It will be understood that the elongated housing need not be rectangular in form, as in the illustrated example, but can be ovoid, tubular, hexagonal, or any shape that permits the extension and retraction of an elongated wick 42, and the contact of the elongated wick 42 with a test strip 20 within the elongated housing 30.

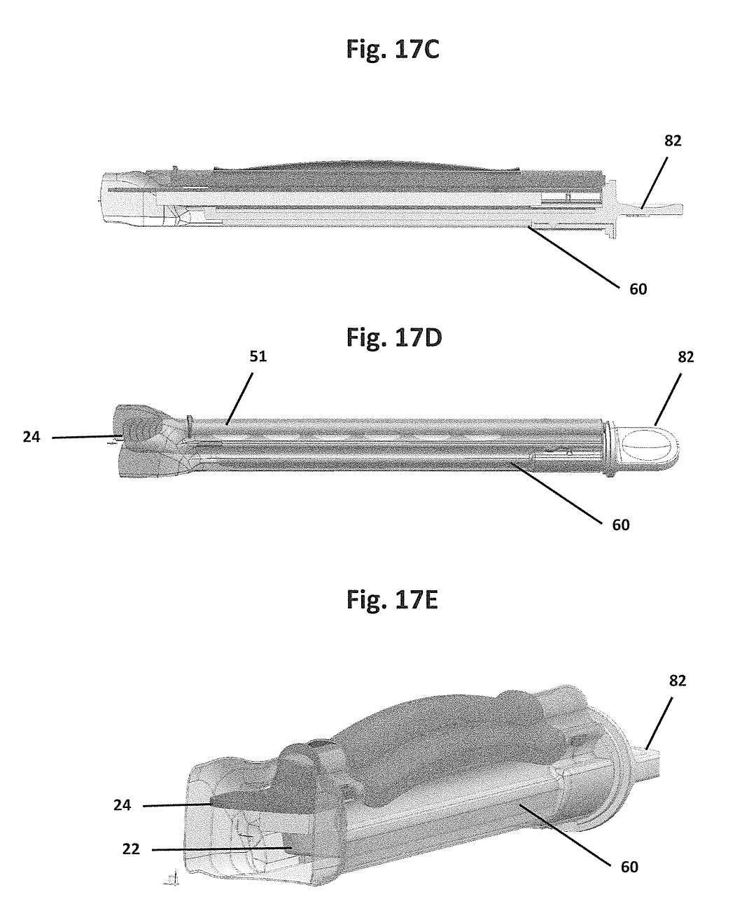

The elongated wick 42 is slidable, by force exerted on the tabs 48, between two positions. In an extended position, a sampling zone 54 of the elongated wick protrudes from the front aperture 44, for exposure to a fluid sample, as shown in FIG. 13B. In a retracted position, at least a portion of the sampling zone 54 is received into the rectangular housing 30, as shown in FIG. 13C. The elongated wick 42 can include a colorimetric strip (not shown) which changes color upon saturation with urine, as previously described for the tubular sample collection device 10. Retraction can also be accomplished with a rod and spring assembly 51, shown in FIGS. 17A and 17D.

A test strip aperture 52, defined in the rear wall 40, admits a test strip 20, preferably supported by a carrier 22, within the elongated housing 30. The test strip 20 includes a detection zone 56 including at least one analytic surface 58, for the detection or measurement of a component of the fluid. When inserted into the elongated housing 30, the detection zone 56 of the test strip 20 is situated below the sampling zone 54 of the elongated wick 42. The test strip 20, or its carrier 22, can rest on the bottom wall 34 of the elongated housing, or it can be supported by a track or ledge (not shown) defined on the bottom wall 34 or the bottom wall 34 and side walls 36 of the elongated housing 30. Alternatively, the extendable sample collection device 60 can be manufactured with a test strip 20 already in place on the bottom wall 34 or on a track or ledge (not shown).

The top wall 32 of the elongated housing 30 is sufficiently depressible to maintain at least the sampling zone 54 of the elongated wick 42 in one of two positions, an outward position distant from the detection zone 56 of the test strip 20, and a depressed, inward position, in contact with the detection zone 56 of the test strip 20 (not shown). In the depressed position, the elongated wick 42 transfers at least a portion of absorbed fluid to the detection zone 56.

As described previously, the detection zone 56 can include multiple analytic areas or sensors 58, which can differ with respect to physical materials, reagents, or both, so that multiple analyses can be accomplished on a single test strip 20. After transfer of the absorbed fluid onto the test strip 20, the test strip is removable from the elongated housing 30, for analysis of a colorimetric, enzymatic, or electrochemical result, or for any required additional processing, such as heating and incubation with additional reagents. In the preferred embodiment, the carrier 22 is withdrawn from the extendable sample collection device 60 and inserted into a separate analyzer device (not shown) that measures the color values of each of the analytic surfaces 58 on the test strip 20 and transmits the colorimetric, enzymatic, or electrochemical data to reporting software residing on a personal computer, laptop, tablet or smart phone (not shown). Embodiments wherein the color of the test strip 20 is not inserted into an analyzer, but rather is read by visual inspection, are less preferable, but are also within the scope of the present invention.

A cleaner/deodorant pad (not shown) can optionally be included to remove residual urine or other fluid from the carrier 22 during its withdrawal, as described previously for the tubular sample collection device 10.

The extendable sample collection device 60 is preferably constructed of a lightweight, flexible thermoplastic, as previously described for the tubular sample collection device 10. The elongated housing 30 is preferably assembled from at least two component injected molded parts. In one example, shown in FIG. 13C, the elongated housing 30 includes two units, an upper unit 90 including the top wall 32, and a lower unit 92, including the bottom wall 34, side walls 36, front wall 38, and rear wall 40. The upper unit 90 and lower unit 92 can be joined by welds, adhesives, hardware such as rivets or nuts and bolts, by a snap fit, or by any other suitable fastening means known in the art (not shown). Preferably, the elongated wick 42 is incorporated into the elongated housing 30 prior to joining of the upper unit 90 to the lower unit 92.

It will be understood that the terms, “front” and “rear”, and “top” and “bottom” are arbitrary terms used to distinguish opposite sides of the tubular sample collection device 10, and of the extendable sample collection device. Components of the invention that are oriented toward the front and rear, or top and bottom, can be collectively transposed without violating the principle or operation of the invention.

Although the exemplary embodiments of the sample collection device of the present invention are directed at the collection of urine and other body fluids, the device is readily adapted, with only minor modifications in structure, for the collection of any fluid that can be absorbed into a wick and transferred to an analytic surface.

The present invention provides a method of collecting a fluid to be analyzed, and transferring the fluid to a detection zone 56 of a test strip 20, including the steps of inserting a test strip 20 into the elongated housing 30, extending a sampling zone 54 of an elongated wick 42 from a retracted position within in the elongated housing 30, to an extended position, exterior to elongated housing 30; exposing the sampling zone 54 to a fluid to be analyzed; absorbing a sample of the fluid to be analyzed into the sampling zone 54; retracting the sampling zone of the elongated wick 42 into the elongated housing, into an outward position, distant from a detection zone 56 of the test strip 20; depressing a top wall 32 of the elongated housing 30; bringing the sampling zone 54 of the elongated wick 42 into contact with the detection zone 56 of the test strip 20 (i.e. contacting the elongated wick 42 to the test strip 20 within the elongated housing 30); and transferring at least a portion of the absorbed fluid sample from the sampling zone 54 to the detection zone 56.

The transferring step is preferably followed by the additional steps of producing a chromatic change at the at least one analytic surface 58 of the test strip 20, removing the test strip from the extendable sample collection device 60, and measuring a colorimetric, enzymatic, or electrochemical result. The step of measuring a colorimetric, enzymatic, or electrochemical result preferably includes the steps of inserting the test strip 20, preferably in the carrier 22, into an analyzing device (not shown); reading a colorimetric, enzymatic, or electrochemical value; producing colorimetric, enzymatic, or electrochemical value data; and transmitting the colorimetric, enzymatic, or electrochemical value data to reporting software residing on a personal computer, tablet, smart phone, or other suitable processing device (not shown).

The present invention provides several advantages. A precise amount of fluid can be transferred from the wick 24 to the test strip 20. No cups, tubes, or excess fluid samples are required to be handled or disposed of. The wick 24, 42 can absorb only 3 to 4 drops of urine, regardless of how long it is held in a fluid stream. The fluid sample is transferred to the assays in the detection zone 56 on the test strip 20 simply by pressing down on the top wall 14 of the sample collection device 10 or by crushing it in one's hand and holding for 1 to 2 seconds. Regardless of position, once pressed, the wick 24, 42 makes contact with each assay pad in the detection zone 56, transferring a relatively precise amount of fluid to begin the chemical reactions. Regardless of position, the wick 24, 42 makes possible transfer of fluid to each assay pad with no cross contamination, which is very common when dipping a test strip into a cup or tube of urine, and which can confound results. When the wick 42 is fully retracted, the sample collection device 60 protects other people from coming into contact with the fluid sample.

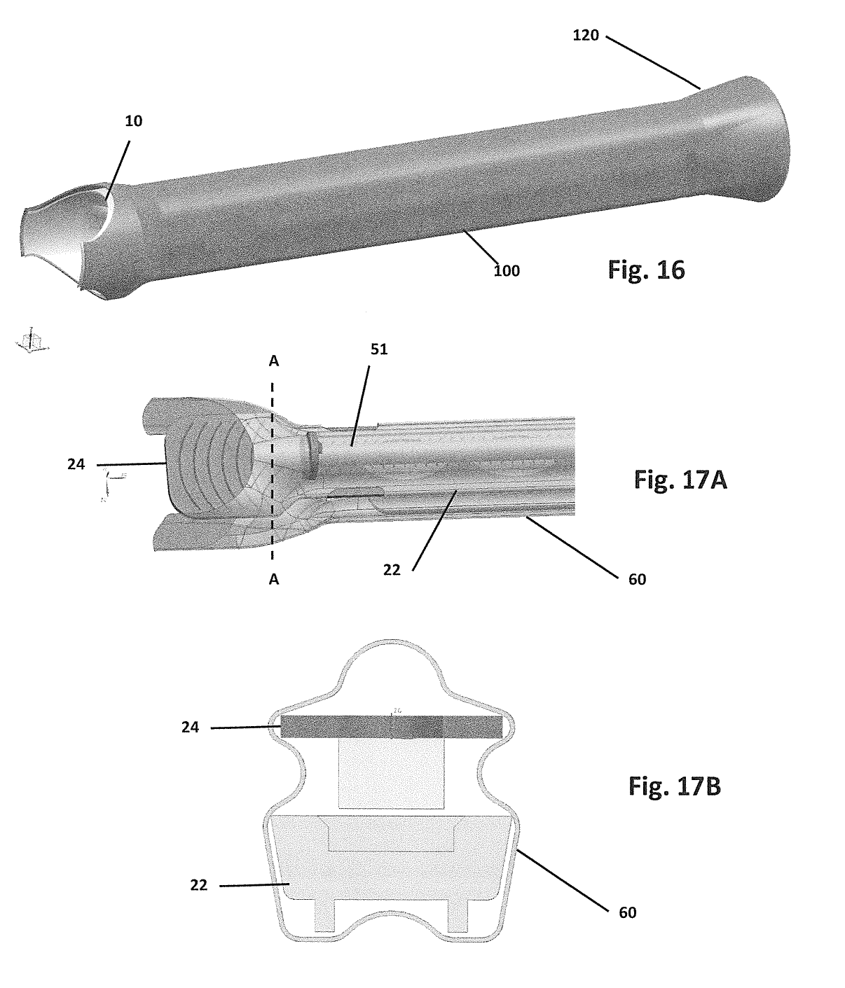

The present invention optionally includes a double biological barrier 100, for preventing the transfer of urine or fluid borne pathogen to users, that covers the entire sample collection device and fits the sample collection device snugly. The double biological barrier 100, as shown in FIGS. 14A, 14B, and 16, includes an inner sleeve 102 and an outer sleeve 104. The inner sleeve 102 is a bag-like structure, of a shape and size that conforms closely to a particular tubular sample collection device 10 or extendable sample collection device 60. The inner sleeve 102 includes at least one open end, termed the inner open end 106. The inner open end 106 accepts the insertion of a sample collection device, 10 or 60, into the interior space 110 of the inner sleeve 102. The inner sleeve 102 acts as a first biological barrier, to prevent the user that is the donor of sample of urine or other fluid, from coming into contact with the sample.

The outer sleeve 104 has essentially the same structure as the inner sleeve 102, but is of sufficiently greater size to permit it to admit the inner sleeve 102 and its enclosed tubular sample collection device, 10 or 60, into its interior space 112. The open end of the outer sleeve 104 is termed the outer open end 108. The outer sleeve 106 forms a second biological barrier to prevent a second user, such as a test administrator, from touching either the sample or any surface previously touched by the user. The term “test administrator” refers to any personnel obtaining a sample collection device from a user. The term includes but is not limited to, a lab technician, nurse, pharmacist, wellness consultant, human resources staffer, early intervention specialist, social worker, or caseworker.

Both the inner sleeve 102 and the outer sleeve 104 are composed of a thin, nonporous, transparent material, such as polyethylene, which is sufficiently deformable to permit a test administrator or other user to depress the top walls, 14 or 32, sufficiently to permit transfer of fluid from a wick, 24 or 42, to a test strip 20.

When used in conjunction with the tubular sample collection device 10, the inner open end 106 of the inner sleeve 102, and the outer open end 108 of the outer sleeve 104, are both oriented to face the first end 25 of the tubular housing 12. This orientation permits the movement of the carrier 22 and test strip 20 into and out of the tubular housing 12, via the inner and outer open ends, 106 and 108 of the inner and outer sleeves, 102 and 104. Similarly, when used in conjunction with the extendable fluid collection device 60, the inner open end 106 of the inner sleeve 102, and the outer open end 108 of the outer sleeve 104, are both oriented to face the test strip aperture 52 of the elongated housing 30. The inner sleeve 102 and the outer sleeve 104 can be separate or can be operatively attached at any suitable point, such as opposite to the ends 106 and 108.

To utilize the double biological barrier 100, a user can handle the outer sleeve 104, extend the wick 24 or 42, and collect a sample (i.e. urinate on the wick 24, 42). With the saturated wick 24 or elongated wick 42 with urine or other fluid, the user optionally presses a flexible section of the outer sleeve 104 to retract the elongated wick 42, and inserts the tubular sample collection device 10 or the extendable sample collection device 60 into the inner sleeve 102 and delivers the resulting assembly to the test administrator. The assembled inner sleeve 102 and sample collection device, 10 or 60, is then inserted into the outer sleeve 104, preferably by the user following the instructions of a test administrator. Optionally, the user can expel the sample collection device 10 from the outer sleeve 104. Prior to analysis of the test strip 20, the test administrator inserts the carrier 22 through the outer open end 108 of the outer sleeve 104, and through the inner open end 106 of the inner sleeve 102, and into the first end 25 of the tubular housing 12 or into the test strip aperture 52 of the elongated housing 30. The test administrator then depresses the top wall 14 of the tubular housing 12, or the top wall 32 of the elongated housing 30, to transfer urine or other fluid to the test strip 20. The test administrator then withdraws the carrier 22 and the test strip 20 for analysis as previously described. After analysis, the test strip 20 is optionally reinserted back into tubular sample collection device 10 or the extendable sample collection device 60, which are still contained within the double biological barrier 100, for disposal. Disposal can be in any suitable sealable disposal container. Preferably disposal is carried out in the original packaging (not shown) of the sample collection device, which is both sealable and equipped with a deodorant strip (not shown), the deodorant strip being activatable during the sealing process. The double biological barrier 100 can also include a flared end 120 which can be pressed to expel the sample collection device 10, 60 from the double biological barrier 100.

There are several advantages to the use of the double biological barrier 100. The user using the sample collection device 10, 60 will contaminate only the outside of the outer sleeve 104 and the tip of the wick 24 assembly. The tip of the wick 24 assembly retracts far enough inside the sample collection device 60 that the technician cannot easily touch the contaminated surface. The sample collection device 10, 60 itself, when expelled from the outer sleeve 104, has riot been touched by the user, and can be safely handled by a technician without necessarily needing another bio-barrier such as gloves. Once returned to its resealable double biological barrier 100, the spent sample collection device 10, 60 creates no significant biohazard.

The previously described methods for collecting a fluid to be analyzed, with either a tubular sample collection device 10 or an extendable sample collection device 60, are readily adapted to include the use of the double biological barrier 100. For each method, the following steps are interposed between the absorbing step and the depressing step: inserting the tubular sample collection device 10 (or the extendable sample collection device 60) through an inner open end 104 of an inner sleeve 102; enclosing the tubular sample collection device 10 (or the extendable sample collection device 60) in the interior space 110 of the inner sleeve 102; inserting the combination of the inner sleeve 102 and tubular sample collection device 10 (or extendable sample collection device 60) through an outer open end 108 of an outer sleeve 104; and enclosing the tubular sample collection device 10 (or the extendable sample collection device 60) in the interior space 112 of the outer sleeve 104.

Most preferably, the sample of fluid in the above description is urine, but other fluids can also be collected, such as, but not limited to, blood, plasma, or any other fluid stream.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1.-23. (canceled)

24. An extendable sample collection device, comprising:
an elongated wick having an extended position from an elongated housing to be exposed to and absorb a fluid sample and a retractable position in said elongated housing, said elongated housing including a top wall being depressible to bring the elongated wick in said retracted position into contact with a test strip situated within said elongated housing.

25. The extendable sample collection device of claim 24, wherein said elongated housing is a hollow rectangular solid shape.

26. The extendable sample collection device of claim 24, wherein said elongated wick partially protrudes through and is slidingly engaged with a front aperture defined in a front wall of said elongated housing.

27. The extendable sample collection device of claim 26, wherein said elongated wick includes two opposite long sides, each said opposite long side having an outward-protruding tab that protrudes through, and is slidingly engaged with, a slot defined along at least part of a length of each of two side walls of said elongated housing.

28. The extendable sample collection device of claim 24, further including a test strip aperture defined in a rear wall for admitting a test strip.

29. The extendable sample collection device of claim 24, wherein said test strip rests on a bottom wall of said elongated housing.

30. The extendable sample collection device of claim 24, wherein said test strip is supported by a carrier within said elongated housing.

31. The extendable sample collection device of claim 30, further including a cleaner/deodorant pad affixed to a bottom wall for removing residual fluid on said carrier.

32. The extendable sample collection device of claim 24, wherein said test strip includes a detection zone including at least one analytic surface for the detection of a component of the fluid.

33. The extendable sample collection device of claim 24, wherein said elongated housing is made from a flexible hydrophobic material.

34. The extendable sample collection device of claim 24, wherein said fluid is urine.

35. The extendable sample collection device of claim 24, further including a double biological barrier for preventing contamination of users including an inner sleeve that conforms closely to said extendable sample collection device including an inner open end for accepting said extendable sample collection device, and an outer sleeve of greater size than said inner sleeve that receives said inner sleeve within an interior space and including an outer open end oriented at said inner open end.

36. A method of collecting a fluid and transferring the fluid to a test strip, including the steps of:
extending an elongated wick from a retracted position within in an elongated housing of an extendable sample collection device to an extended position exterior to the elongated housing;
exposing the elongated wick to a fluid to be analyzed;
absorbing a sample of the fluid to be analyzed into the elongated wick;
retracting the elongated wick into the elongated housing;
contacting the elongated wick to a test strip within the elongated housing; and
transferring fluid from the elongated wick to the test strip.

37. The method of claim 36, wherein said exposing step is further defined as exposing a sampling zone of the elongated wick to the fluid to be analyzed.

38. The method of claim 37, wherein said contacting step is further defined as depressing a top wall of the elongated housing, bringing the sampling zone of the elongated wick into contact with a detection zone of the test strip, and transferring at least a portion of the absorbed fluid sample from the sampling zone to the detection zone.

39. The method of claim 36, further including the steps of producing a change chosen from the group consisting of chromatic, enzymatic, and electrochemical of at least one analytic surface of the test strip, removing the test strip from the extendable sample collection device, and measuring a test result chosen from the group consisting of a colorimetric test result, an enzymatic test result, and an electrochemical test result.

40. The method of claim 36, wherein said step of measuring a test result further includes the steps of inserting the test strip into an analyzing device, reading a value chosen from the group consisting of a colorimetric value, an enzymatic value, and an electrochemical value, producing value data, and transmitting the value data to reporting software.

41. The method of claim 36, further including, after said contacting step, the step of wiping a carrier housing the test strip and removing residual fluid.

42. The method of claim 36, wherein said admitting step further includes the step of indicating that the wick is saturated with fluid and ceasing collection of fluid.

43. The method of claim 36, wherein the fluid is urine.

44. The method of claim 36, further including, after said absorbing step, the steps of inserting the extendable sample collection device in an inner sleeve of a double biological barrier, and inserting the inner sleeve in an outer sleeve of the double biological barrier.

45. (canceled)

46. (canceled)

47. (canceled)

48. A sampling device comprising:
a fluid collection device comprising a housing having a hollow interior;
a wick within the interior of the housing;
a test strip within the interior of the housing; and
a double biological barrier surrounding the fluid collection device, the double biological barrier comprising an inner sleeve surrounding the fluid collection device and an outer sleeve surrounding the inner sleeve.

49. The sample device of claim 48, wherein the housing is elongate and radially compressible from a tubular shape to a flattened shape, and wherein the wick and the test strip are distanced from each other when the housing is in the tubular shape and in contact when the tubular housing is in the flattened shape.

50. The sample device of claim 49, wherein the housing is radially compressible from the tubular shape to the flattened shape by application of a pressure on a wall of the housing and wherein the housing returns to the tubular shape upon removal of the pressure.

51. The sample device of claim 48, wherein the wick is movable between a first retracted position where the wick is within the interior of the housing and a second extended position where the wick is outside of the housing.

52. The sample device of claim 48, wherein the test strip is reversibly insertable into and removable from the interior of the housing.

53. The sample device of claim 48, wherein the inner sleeve comprises an inner open end and the outer sleeve comprises an outer open end, and wherein the inner and outer sleeves are nested such that the inner open end is oriented at the outer open end.

54. The sample device of claim 48, wherein the inner sleeve and the outer sleeve are made of a thin, nonporous, transparent material.

55. The sample device of claim 48, wherein the outer sleeve is removable from the inner sleeve while the inner sleeve remains surrounding the fluid collection device.
